(19) United States
(12) Patent Application Publication
(10) Pub. No.: US 2017/0273821 A1
(43) Pub. Date: Sep. 28, 2017

(54) Array Of Cutaneous Patches For Delivery And Heating Of Personal Care Products

(71) Applicant: ELC Management LLC, Melville, NY (US)

(72) Inventors: Herve F. Bouix, New York, NY (US); Christophe Jacob, Roussillon (FR)

(21) Appl. No.: 15/076,791

(22) Filed: Mar. 22, 2016

(51) Int. Cl. A61F 7/00

(52) U.S. Cl. CPC A61F 7/007; A61F 2007/0077; A61F 2007/0052

(57) ABSTRACT

An array of heating patches for placement on the skin, especially the skin of the face. Each patch has a heating circuit incorporated therein, and the patches are electrically connected to each other in series and/or parallel. Electrical energy, and optionally, digital information, may be supplied to the patches through one or more cables that originate in an appropriate electrical connector. Each patch has two layers; a disposable layer and a reusable layer.

### FIELD OF THE INVENTION

The invention is in the field of cosmetic patches for delivery of one or more personal care products. Specifically, the invention comprises adhesive patches that are able to heat a product, as well as the skin of a user.

### BACKGROUND

Nowadays, personal care companies seek to attract consumers by incorporating a source of heat into the cosmetic or personal care experience. The heat, by itself, may provide benefits to skin or hair. Or, the heat may activate a product. Among the obstacles to development of heated systems is the need to supply a portable power source. The power source must be able to supply power to use the system for repeated applications. Often this means that the power source is heavy, and adds a lot of cost to each unit produced. The power source quickly depletes, and needs to be replaced or recharged. All of these have been obstacles to development and barriers to entry in the marketplace.

Sometimes it is desirable to supply a personal care product at an elevated temperature. The benefits of the heat product include, a pleasurable sensation, the ability to activate an active ingredient that lies dormant in the product, improved penetration of product through the skin barrier.

### OBJECTIVES

A main objective is to provide a simple, economical means for heating a product as it is being delivered to the skin.

Another objective is to provide a simple means for heating the skin at a site of application of a cosmetic or personal care product.

### SUMMARY

In a preferred embodiment, the present invention is a set of heating patches for placement on the skin, especially the skin of the face. Each patch has a heating circuit incorporated therein, and the patches are electrically connected to each other in series and/or parallel. Electrical energy, and optionally, digital information, may be supplied to the patches through one or more cables that originate in an appropriate electrical connector. One such connector is a USB style connector. Each patch has two layers; a disposable layer and a reusable layer. During use, the disposable layer adheres to the skin, and the reusable layer, which comprises the heating circuit, adheres to the disposable layer. The disposable layer is suitable for being impregnated with a product, which is then held against the skin, while being heated. A protective film may cover the disposable layer prior to use. The heating patch system may comprise various electronic hardware and software elements, such as: a timer, a digital memory structure, a digital operating system, and programming code.

### DESCRIPTION OF THE FIGURES

FIG. 1 depicts one embodiment of an array (5) of cutaneous patches (1) for delivery and heating of personal care products.

FIG. 2 depicts a top perspective view of a disposable adhesive patch (2).

FIG. 3 depicts a bottom perspective view of a disposable adhesive patch (2).

FIG. 4 depicts two reusable heating pads (3) connected by a power cable (4d).

FIG. 5 an array of reusable heating pads (3) connected by a power cable (4d)

FIG. 6 depicts a bottle (6) of personal care product that has a dropper applicator (7).

FIG. 7 depicts product (P) being applied to a disposable adhesive patch (2).

FIG. 8 shows an array (5) of cutaneous patches (1) being used on the face of a user.

### DETAILED DESCRIPTION

By “comprise”, we mean that a group of elements is not limited to those explicitly recited, but may or may not include additional elements.

“Personal care product” includes products that benefit the skin and hair, as well as makeup for the skin or hair. The term is intended to have a broad definition.

### Introduction

Of course, in the developed world and beyond, computing devices have saturated the home and office. And, with advances in the technology of portable devices (for example, cell phones, media players, game consoles, digital cameras and computing devices), the availability of power sources “on the go” has increased dramatically. And, as consumer electronics continue to improve, the number of electronic devices that are able supply the power required by the present invention will only increase. Furthermore, the number and types of locations where a user can recharge an electronic device has risen dramatically. For example, commuter busses, trains and airplanes are commonly equipped with power outlets as a convenience for their riders, whose device battery may be depleted. More and more, coffee shops and other business establishments must provide their customers with convenient free power, or risk losing their business. The present invention takes advantage of this increasing access to low voltage power.

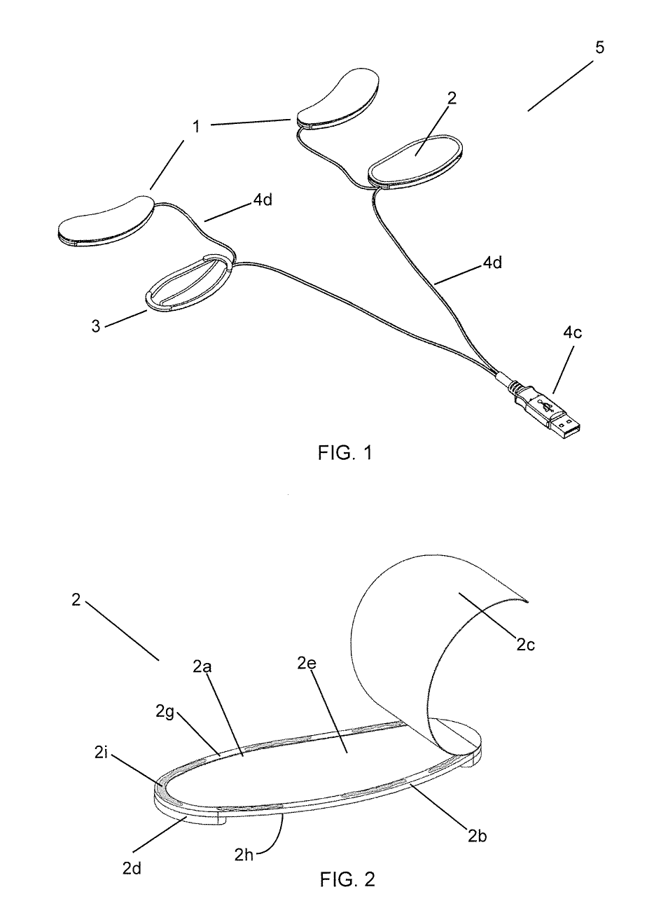

Referring to FIG. 1, a set of cutaneous patches (1) is used to heat and deliver a personal care product to the skin of a user. Each cutaneous patch (1) comprises a disposable adhesive patch (2) temporarily joined to a reusable heating pad (3). The reusable heating pads are joined by power cables (4d) to form an array (5) of reusable heating pads (3). Electrical power enters and leaves the array at a power connector (4c). In some preferred embodiments of the invention, the power cables and power connector are able to transmit data.

### The Disposable Adhesive Patch

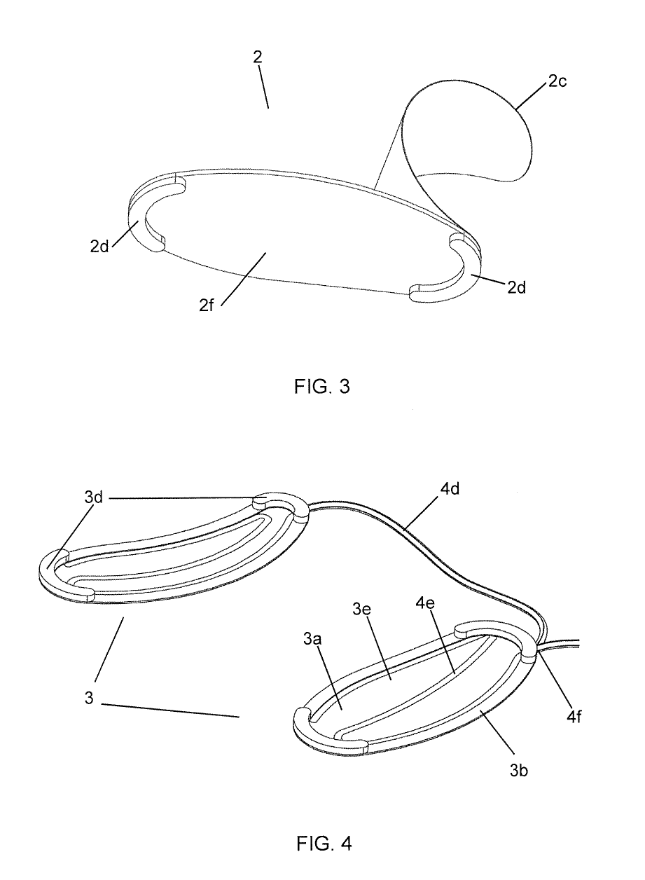

Referring to FIG. 2, the disposable adhesive patch (2) comprises an substrate (2a) which is held taut by a frame (2b). Prior to use, the top side (2e) of the substrate is covered by a removable tab (2c). Referring to FIG. 3, the bottom side (2f) of the substrate (2a) is able to be detachably connected to the top side (3e) of a reusable heating pad (3, see below). Various types of detachable fasteners may be suitable. A first portion of a detachable fastening mechanism will be located on the bottom side (2f) of the substrate (2a), while a complementary second portion is located on the top side of the of the reusable heating pad (3). For example, the bottom side (2f) of the substrate (2a) may have one or more halves (2d) of a hook-and-loop fastener (such as that commonly marketed under the Velcro® brand) permanently affixed thereon. The hook or loop fastener portions (2d) on the bottom side (2f) of the substrate (2a) cooperate with complementary halves located on the top side (3e) of the reusable heating pads (3).

The substrate (2a) is able to be impregnated by a personal care formulation. The substrate is preferably an absorbent material, thin and flexible so that it may contour to the surface to which it is applied. Preferably, the substrate is an absorbent, cellulosic tissue paper having significant wet strength. Just prior to use, the top side (2e) of the tissue paper substrate will be loaded with a dose of personal care product. Thus, it is important that the substrate have a wet strength that is great enough to prevent tearing due to the weight of the product and handling of the disposable patch. Identifying a whole range of suitable strong, absorbent materials is not difficult. The perimeter of the substrate (2a) is attached to the bottom side (2h) of a frame (2b). The frame is plastic or elastomeric and sufficiently rigid to hold the substrate (2a) taut, but also flexible so that it may contour to the surface to which it is applied. The top side (2g) of the frame is coated with a reusable adhesive (2i, such as a pressure sensitive adhesive) and, prior to use, the tab (2c) is removably adhered to the frame. The adhesive should be compatible with human skin, i.e. non-allergenic, non-toxic and non-irritating. The tab protects the tissue paper substrate (2a) from damage and dirt. The tab is removed prior to using the disposable adhesive patch (2), and the reusable adhesive on the frame (2b) is used to adhere the cutaneous patch (1) to the skin. The hook or loop fastener portions (2d) on the bottom side (2f) of the substrate (2a) will cooperate with complementary halves located on the reusable heating pads (3).

### The Reusable Heating Pad

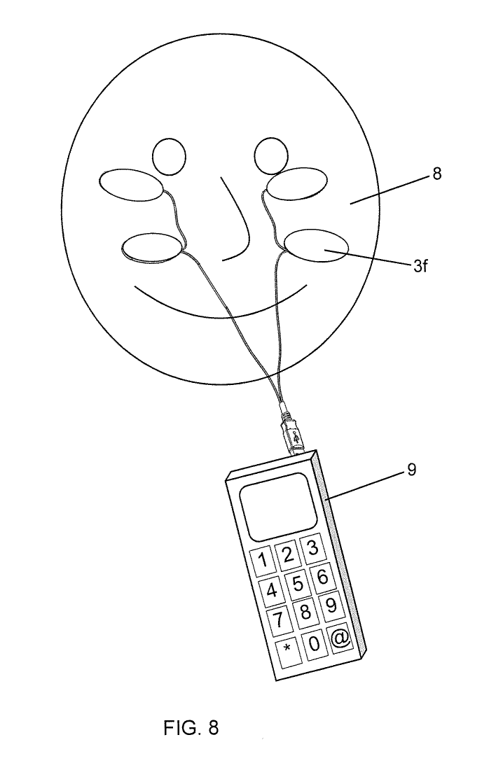

Referring to FIG. 4, each reusable heating pad (3) comprises a substrate (3a), preferably a non-absorbent substrate, surrounded by perimeter (3b). Examples of non-absorbent substrate material include plastic films, such as PET, and coated papers. The non-absorbent substrate is durable and flexible. The top side (3e) of each non-absorbent substrate is able to be detachably connected to the bottom side (2f) of the disposable adhesive patches (2). Various types of detachable fasteners may be suitable. A second portion of a detachable fastening mechanism is located on the top side of the non-absorbent substrate, while a complementary first portion is located on the bottom side of the disposable adhesive patch. For example, each non-absorbent substrate (3a) may comprise one or more halves (3d) of a hook-and-loop fastener (such as that commonly marketed under the Velcro® brand) permanently affixed thereon. The hook or loop fastener portions (3d) on the top side (3e) of the non-absorbent substrate (3a) cooperate with complementary halves located on the bottom side (2f) of the disposable adhesive patches (2). Thus, it is preferable if the disposable adhesive patches and the reusable pads have the same number of hook-and-loop halves, specifically positioned to meet its complementary half. It is also preferable if the disposable patches and reusable pads are a similar size and shape. The bottom side (3f) of the reusable heating pad (3) is the surface that will be exposed during use (see FIG. 8).

### The Heating Circuit

A heating portion (4e) of an electric heating circuit (4) is located on the top side (3e) of the non-absorbent substrate (3a) of each reusable heating pad (3). This heating portion of the electric circuit is able to generate heat when electricity is flowing though the portion. In a preferred embodiment, the heating portion (4e) is formed as a continuous electric path that loops over the pad to generate heat evenly over the whole pad. For example, in FIG. 4, the heating portion is shown to make three passes across the non-absorbent substrate (3a). The electric path starts and ends near the perimeter (3b) of the pad, where it connects to one or more power (or power/data) cables (4d) that carry electrical power to and from the reusable pads (3). This connection is labelled (4f) in FIG. 4. In this way, a number of reusable pads may be connected in series and/or parallel, joined by the requisite number of power cables (4d), to form a complete array (5) of reusable heating pads (3). The power cables (4d) terminate at a power connector (4c).

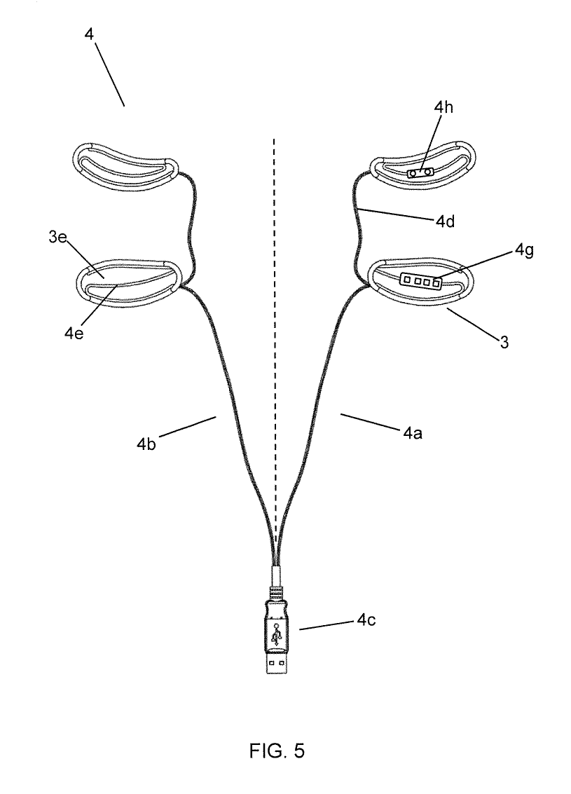

FIG. 5 shows one embodiment of such an array, having four reusable heating pads (3), joined by four cables (4d). The four reusable pads of FIG. 5 could have been arranged in series, but in preferred embodiments of the invention, half of the pads will be used on either side of the face, so separating the pads into a right main branch (4a) and a left main branch (4b) is convenient for the user. Within the right and/or left main branches, there may be sub-branches. The main branches, however, begin and end at a power or power/data connector (4c). Preferred types of connectors include any of those in the USB family of connectors. Cylindrical power connectors, such coaxial connectors may also be useful, as well as other standardized connectors. Of course, plug converters may be used in the usual way. For example, an adapter may be used to convert a USB connector into an AC power plug, or to convert a USB connector into an automobile power outlet (i.e. a cigarette lighter-style outlet). Taken together, the power connector (4c), power cables (4d), and heating portions (4e) define an electric heating circuit (4) of the invention.

In preferred embodiments, the cutaneous patches (1) are powered by connecting a USB-type connector (4c) to a device that is able to supply low voltage power. Such devices may have a complementary USB jack or require the use of an adapter. Such devices may include a desk top computer, automobile console, courtesy outlet in a bus, train or plane, a cell phone, a tablet computing device, or other device that can provide low voltage electric power. Once plugged in, a heating circuit is completed (i.e. closed) and electricity will flow across the non-absorbent substrates (3a) of each reusable heating pad (3). While this is happening, heat is generated by the heating portions (4e) of each reusable heating pad. The product on the disposable patch (2) is heated, and the skin around the reusable pad is also heated. When the USB jack is unplugged, then the heating circuit is opened, and heating stops.

In preferred embodiments, the heating portion (4e) of the reusable heating pad (3) is implemented as one or more Flexible Printed Circuits (FPC). One or more FPCs may be incorporated into the invention by adhering one or more prefabricated FPCs to the top side (3e) of the non-absorbent substrate (3a) using an adhesive. Examples of prefabricated FPCs include those manufactured by Minco (Minneapolis, Minn.) and those manufactured by Tempco (Wood Dale, Ill.). Alternatively, one or more FPCs may be printed directly onto the top side (3e) of the non-absorbent substrate (3a). One of the most cost effective methods of depositing conductor material onto a flexible substrate uses conventional ink printing techniques. Polymer thick film (PTF) inks may be applied to a substrate using various technologies known from conventional ink printing, such as screen printing, flexography, gravure, offset lithography, and inkjet printing. Printed PTF electronics is a comparatively low cost, high volume process. PTF inks are a mixture of a polymer binder (i.e. polyester, epoxy, acrylic) and a granulated conductive material such as silver, resistive carbon or both. The ink may be applied directly to the substrate (3a) without a separate adhesive. A preferred substrate for this application is PET. Although silver and carbon polymer thick-film (PTF) inks are the most common inorganic inks, various companies offer an assortment of other ink types, such silver chloride, silver carbon, platinum, gold, and phosphors. Organic ink types include conductive polymers such as poly(aniline) and poly(3,4-ethylene dioxitiophene), doped with poly(styrene sulfonate). Polymer semiconductors include poly(thiopene)s like poly(3-hexylthiophene) and poly(9,9-dioctylfluorene co-bithiophen). Those inks that when cured offer greater flexibility and scuff resistance are generally preferred.

Of particular note for the present invention are positive thermal coefficient (PTC) inks, such as PTC-614, PTC-842, PTC-921 and PTC-922 inks available from Conductive Compounds (Hudson, N.H.). These inks are suitable for low DC voltage applications, and are self-regulating, which means that once a certain temperature is reached, the ink is able to maintain a temperature range (for example, 45° C. to 50° C. or 50° C. to 60° C. or 60° C. to 70° C.) without a feedback loop.

In addition to the heating portion (4e), the top side (3e) of the non-absorbent substrate (3a) may incorporate bulkier non-printed electronic elements. Technically speaking, there is nothing that prevents the incorporation of such elements in the printed circuit; it's a question of cost and convenience. Electronic elements that may be useful include thermistors, timers, voltage regulators, capacitors, resistors, LEDs, integrated circuit chips, logic gates, etc.

In preferred embodiments, the time to heat a reusable heating pad (3) and the product on the disposable adhesive patch (2) to at least 45° C. is 3 minutes or less; more preferred is 2 minutes or less. Some USB specifications fix the voltage at 5 V±5% (4.75 V to 5.25 V). Thus, by adjusting the resistance of the heating portion (2), the desired power conversion rate may be achieved. The electrical resistance of the heating element can be adjusted by the composition of the ink, by the amount of ink deposited, and by the cross sectional area of the deposited ink. A useful range of heating element resistance is about 1 Ω to about 15 Ω; preferred is 2 Ω to 10 Ω; more preferred is 3 Ω to 5 Ω. For example, if the heating element resistance is between about 2.5 Ω and 5 Ω, then a 5 V power supply produces a current of about 1 A to 2 A, and power is provided at about 5 W to 10 W, which is sufficient to heat a dose of product to 45° C. in 2-3 minutes. The self-regulating nature of the positive thermal coefficient ink may be used to prevent the temperature from increasing beyond any point one wishes to define, such as 50° C., or 60° C. or 70° C., etc., even if the circuit is left on for an extended period of time.

### The Product

Prior to use, the disposable substrate (2a) may be loaded with product. Virtually any form of personal care product can be used with the present invention, including creams, lotions, powders, pastes, gels, serums, loose liquids, etc. Virtually any type of personal care product can be used with the present invention, including, but not limited to skin treatment products, color cosmetics, hair growth and hair removal products. Skin treatment products, for example, include products for: wrinkle treatment, DNA repair, plumping, moisturizing, whitening, acne treatment, after sun care, anti-oxidation, anti-inflammation, collagen boosting, exfoliation, cleansing, and barrier repair. The present invention is particularly suited for targeted delivery of active ingredients, especially when delivery of those actives will be facilitated by heat. A dose of product may be supplied to the disposable substrate (2a) from a tube, dropper, aerosol container, wand applicator, etc. Alternatively, a user may transfer a dose of product from a container to the disposable substrate with a finger.

### Methods of Use

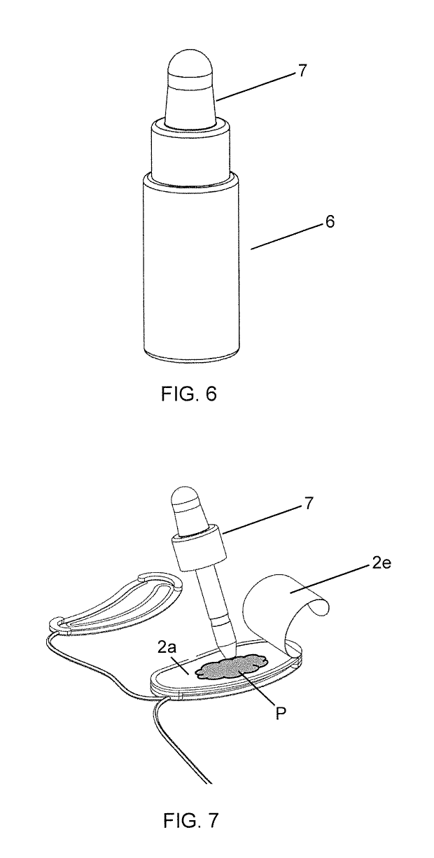

We describe one method of treating the skin of a user with a personal care product and heat. In use, a user will have an array (5) of reusable heating pads (3). Separately, the user will have set of disposable patches (2), and a container of personal care product. The cutaneous heating array could be used without product, as a heat treatment for the skin, but the invention is best practiced in conjunction with a personal care product. In the example of FIG. 6, the container is a bottle (6) having a dropper applicator (7). First, a user will affix a disposable patch (2) to each reusable heating pad (3), via the hook and loop fasteners (2d, 3d), to form an array of adhesive patches (5). One by one, the tabs (2c) are peeled off of the disposable patches (2), exposing the reusable adhesive (2i) along the frame (2b). A dose of product is applied to the top side (2e) of the absorbent substrate (2a). FIG. 7 shows the product (P) being applied by a dropper (7). Thereafter, the top side of each absorbent substrate is pressed against the skin. This can be anywhere on the body, but the face (8) will be the most common site of treatment (FIG. 8). The absorbent substrate adheres to the skin because of the reusable adhesive (2i). Each adhesive patch is positioned where treatment is most desired. All or only some of the patches may be used during any one treatment. Once all of the patches are adhered to the skin, the power connector (4c) is plugged into a power source (9). The power source may be, for example, a cell phone, media player, game console, digital camera, a computing device, such as a laptop computer or other device. In FIG. 8, the power source (9) is a cell phone. The heating begins immediately. Heat from the heating portion (4e) passes through the absorbent substrate (2a). The product (P) is heated to a desired maximum temperature within three minutes or less. The user allows the product to each a predetermined temperature, and then continues the treatment for several minutes or hours, preferably as directed by a set of product instructions. For example, after reaching a predetermine temperature, a user may wear the cutaneous patches (1) for at least 1 minute, preferably at least 5 minutes, more preferably at least 10 minutes. Heat generation may be discontinued at any time by unplugging the power connector (4c). When finished, the cutaneous patches (1) are removed from the skin. The disposable adhesive patches (2) are separated from the reusable heating pads (3), and discarded. The array (5) of reusable heating pads (3) is ready to be used again.

Heating a dose of product (P) on the disposable adhesive patch (2) is unlike heating a larger quantity of product in a reservoir. In that situation, the volatile components of the product are lost more quickly each time the product in the reservoir is heated and exposed to the ambient atmosphere. Product dry-out is a serious problem to the marketing of such products. However, in the present invention, when used as directed, product dry-out is not a realistic problem, because the product is being consumed as it is heated. Thus, even when the dose of product comprises volatile ingredients, it is unlikely to be significantly deteriorated by heating.

### Some Optional Features

In some preferred embodiments, a shut off timer (4g) may be included in the heating circuit (4), in case a user accidentally leaves the circuit on beyond a predetermined length of time. Optionally, an indicator that tells the user when a certain temperature has been reached may be included in the heating circuit. The indicator may be a thermo-chromic material incorporated into the non-absorbent substrate (3a), that turns a certain color when a set temperature has been reached.

In some embodiments, a USB power connector (4c) as described herein is preferred. By connecting the USB connector to a device with internet or other network access, it may be possible to transfer data to and from the array (5) of reusable heating pads (3). For example, one or more heating portions (4e) of the heating circuit (4) may comprise an integrated circuit element (4h) having programmed instructions. When the heating circuit is completed by plugging the USB plug into an internet enabled device, the coded instructions of the integrated circuit element may pass to the device, to initiate all sorts of informational and media experiences normally associated with such devices. For example, when the USB plug is inserted into an internet enabled device, a web site may be launched having content that complements the use of the invention. For example, a video of a beauty advisor who offers advice and information about the use of one or more products may appear, or a promotional offer for a product may be made. The experience can be interactive, so the user can identify which product she is sampling and the appropriate content can be downloaded to the users device. Another type of user interaction would allow the user to control her own treatment, including adjusting the temperature. This could be done with the assistance of a specifically designed “app” that runs on the USB-connected device.

### A Kit

The present invention encompasses a kit comprising an array (5) of two or more reusable heating pads (3) joined by USB-type power/data cables (4d), which terminate in an USB-type connector (4c). The array may comprise as many heating pads as is practical, considering the number that a user may find unwieldy and the amount of available power to run each heating pad. Typically, an array may comprise from 2-12 reusable heating pads, preferably 4-10, more preferably 4-8. The kit also comprises a number of disposable adhesive patches (2), at least as many there are reusable heating pads. Preferably, the kit comprises at least twice as many disposable adhesive patches as there are reusable heating pads, or more preferably, at least three times as many. For example, if in the kit, the array (5) of reusable heating pads (3) comprises four such heating pads, then a kit will comprise at least four disposable adhesive patches (2), but preferably comprises twenty-eight disposable adhesive patches (for daily use for one week), or more preferably one-hundred twenty disposable adhesive patches (for daily use for one month). In kit with eight reusable heating pads, a one month supply of disposable adhesive patches is two-hundred forty. The kit also comprises at least one personal care product in a container. The array of reusable heating pads, disposable adhesive patches and one or more containers of personal care product are packaged for sale as a unit.

What we claim is:

1. An array of two or more cutaneous patches (1), each cutaneous patch comprising a reusable heating pad (3) detachably connected to a disposable adhesive patch (2):
each disposable adhesive patch (2) comprises:
a frame (2b) having a top side (2g) and a bottom side (2h);
an adhesive that is compatible with human skin located on the top side (2g) of the frame (2b)
a substrate (2a) attached to the bottom side (2h) of a frame (2b), the substrate having a top side (2e) and a bottom side (2f);
a first portion (2d) of a detachable fastening mechanism located on the bottom side (2f) of the substrate (2e);
each reusable heating pad (3) comprises:
a substrate (3a) having a top side (3e);
a second portion (3d) of the detachable fastening mechanism located on the top side (3e) of the substrate (3a); and
a heating portion (4e) located on the top side (3e) of the substrate (3a), that generates heat when electricity is flowing though the portion;
wherein the heating portions (4e) of the reusable heating pads (3) are connected in series and/or parallel via electronic cables, and the cables terminate at a power connector (4c).

2. An array of cutaneous patches (1) according to claim 1 wherein the substrate (2a) is an absorbent material, substrate (3a) is a non-absorbent material, and wherein each disposable adhesive patch (2) further comprises a tab(2c) that cover the substrate (2a), the tab being removably adhered to the top side (2g) of the frame (2b) by the adhesive.

3. An array of cutaneous patches (1) according to claim 1 wherein the detachable fastening mechanism is a hook-and-loop fastener.

4. An array of cutaneous patches (1) according to claim 1 wherein the heating portions (4e) of the reusable heating pads (3) are formed as a continuous electric path that loops over the pad.

5. An array of cutaneous patches (1) according to claim 1 wherein the heating portion (4e) of each reusable heating pad (3) is a Flexible Printed Circuit (FPC).

6. An array of cutaneous patches (1) according to claim 5 wherein the circuit is printed on the top side (3e) of the substrate (3a), in a positive thermal coefficient ink.

7. An array of cutaneous patches (1) according to claim 1 wherein the power cables (4d) and power connector (4c) are able to transmit data.

8. An array of cutaneous patches (1) according to claim 7 wherein the power connector (4c) is a USB type connector.

9. An array of cutaneous patches (1) according to claim 8 wherein one or more heating portions (4e) in the reusable heating pads comprise an integrated circuit element (4h) having programmed instructions.

10. An array of cutaneous patches (1) according to claim 1 wherein the array comprises from 2-12 reusable heating pads (3).

11. A kit comprising:
an array of two or more reusable heating pads (3), each reusable heating pad (3) comprising:
a substrate (3a) having a top side (3e);
a second portion (3d) of a detachable fastening mechanism located on the top side (3e) of the substrate (3a); and
a heating portion (4e) located on the top side (3e) of the substrate (3a), that generates heat when electricity is flowing though the portion;
wherein the heating portions (4e) of the reusable heating pads (3) are connected in series and/or parallel via electronic cables, and the cables terminate at a power connector (4c);
at least as many disposable adhesive patches (2) as there are reusable heating pads 93), each disposable adhesive patch comprising:
a frame (2b) having a top side (2g) and a bottom side (2h);
an adhesive that is compatible with human skin located on the top side (2g) of the frame (2b)
a substrate (2a) attached to the bottom side (2h) of a frame (2b), the substrate having a top side (2e) and a bottom side (2f);
a first portion (2d) of the detachable fastening mechanism located on the bottom side (2f) of the substrate (2e); and
one or more containers (6) of personal care product.

12. A kit according to claim 11 having 4-8 reusable heating pads (3) and 28-240 disposable adhesive patches (2).

13. A method of treating skin with a personal care product and heat, comprising the steps of:
having a kit according to claim 11;
affixing a disposable adhesive patch (2) to each reusable heating pad (3);
exposing the reusable adhesive along the frame (2b) by peeling the tabs (2c) off of the disposable adhesive patches (2);
applying a dose of product to the top side (2e) of the disposable adhesive patches (2);
adhering each disposable adhesive patches (2) to the skin;
plugging the power connector (4c) into a power source (9);
allowing the product to reach a predetermined temperature;
continuing the treatment for at least one minute;
unplugging the power connector (4c);
removing each disposable adhesive patch (2) from the skin;
separating the disposable adhesive patches (2) from the reusable heating pads (3).
